(19) United States
(12) Patent Application Publication
(10) Pub. No.: US 2008/0249305 A1
(43) Pub. Date: Oct. 9, 2008

(54) Novel imidazole based heterocycles

(76) Inventors: David J. Calderwood, Framingham, MA (US); Kristine E. Frank, Worcester, MA (US); Patrick Betschmann, Bueren So (CH); Eric C. Breinlinger, Charlton, MA (US); Richard W. Dixon, Jefferson, MA (US)

Correspondence Address: ABBOTT BIORESEARCH, 100 RESEARCH DRIVE, WORCESTER, MA, 01605-4314, US

(21) Appl. No.: 12/079,510

(22) Filed: Mar. 27, 2008

Related U.S. Application Data
(60) Provisional application No. 60/920,246, filed on Mar. 27, 2007.

(51) Int. Cl. C07D 487/04

(52) U.S. Cl. 544/295

(57) ABSTRACT

The present invention is directed to novel imidazopyrazine compounds useful as kinase inhibitors and as such would be useful in treating certain conditions and diseases, especially inflammatory conditions and diseases and proliferative disorders and conditions, for example, cancers.

### CROSS REFERENCE TO RELATED APPLICATION

This application claims priority to U.S. Provisional Application Ser. No. 60/920,246 filed on Mar. 27, 2007, the contents of which are incorporated herein in its entirety.

### BACKGROUND OF THE INVENTION

Protein phosphorylation, at specific amino acid residues, is important for the regulation of many cellular processes including cell cycle progression and division, signal transduction, and apoptosis. The phosphorylation is usually a transfer reaction of the terminal phosphate group from ATP to the protein substrate. The specific structure in the target substrate to which the phosphate is transferred is a tyrosine, serine or threonine residue. Since these amino acid residues are the target structures for the phosphoryl transfer, these protein kinase enzymes are commonly referred to as tyrosine kinases or serine/threonine (S/T) kinases. The phosphorylation reactions, and counteracting phosphatase reactions, on the tyrosine, serine and threonine residues are involved in countless cellular processes that underlie responses to diverse intracellular signals, regulation of cellular functions, and activation or deactivation of cellular processes. A cascade of protein kinases often participate in intracellular signal transduction and are necessary for the realization of cellular processes. Because of their ubiquity in these processes, the protein kinases can be found as an integral part of the plasma membrane or as cytoplasmic enzymes or localized in the nucleus, often as components of enzyme complexes. In many instances, these protein kinases are an essential element of enzyme and structural protein complexes that determine where and when a cellular process occurs within a cell. Given the importance and diversity of protein kinase function, it is not surprising that alterations in phosphorylation are associated with many diseases such as cancer, diabetes, inflammation, and hypertension.

The identification of effective small molecules that specifically inhibit protein kinases involved in abnormal or inappropriate cell proliferation, signaling, differentiation, protein production, or metabolism is therefore desirable. In particular, the identification of methods and compounds that specifically inhibit the function of kinases that are involved in immune modulation or proliferative disorders.

The present invention provides novel compounds that inhibit one or more S/T kinase or receptor or non-receptor tyrosine kinase. The compounds of the present invention affect cytokine inhibitory activity.

Cytokine mediated diseases and cytokine inhibition, suppression and antagonism are used in the context of diseases or conditions in which excessive or unregulated production or activity of one or more cytokine occurs. Examples of such cytokines are tumour necrosis factor alpha (TNFα), interleukin-1 (IL-1), interleukin-6 (IL-6) and interleukin-8 (IL-8). There remains a need for compounds which are useful in treating cytokine mediated diseases, and as such, inhibit, suppress or antagonize the production or activity of cytokines such as TNF, IL-1, IL-6 and IL-8.

The p38 MAP kinase (p38, also known as CSBP or SAPK) signaling pathway has been reported to be responsible for the expression of pro-inflammatory cytokines (such as TNF, IL-1, IL-6, IL-8) that are elevated in many inflammatory and auto-immune diseases (see J. C. Lee, Nature Reviews Drug Discovery 2003, 2, 717-726 and references cited therein). This pathway has been shown to be activated by cellular stressors, such as osmotic shock, UV light, free radicals, bacterial toxins, viruses, cytokines, chemokines and in response, mediates the expression of several cytokines including, but not limited to, TNF, IL-1, IL-6 and IL-8. In cells of myeloid lineage, such as macrophages and monocytes, both IL-1 and TNFα are transcribed in response to p38 activation. Subsequent translation and secretion of these and other cytokines initiates a local or systemic inflammatory response in adjacent tissue and through infiltration of leukocytes. While this response is a normal part of the physiological response to cellular stress, acute or chronic cellular stress leads to the excess or unregulated expression of pro-inflammatory cytokines. This, in turn, leads to tissue damage, often resulting in pain and debilitation. (see G. Panayi, N Engl J Med 2001, 344(12), 907; J. Smolen Nature Reviews Drug Discovery 2003, 2, 473 and references cited therein). The four known isoforms of p38 MAP kinase (p38 α, β, γ, δ) each showing different expression levels, tissue distributions and regulation, support the concept that they are involved in the etiology of many diseases.

Many solid tumours increase in mass through proliferation of malignant cells and stromal cells, including endothelial cells. In order for a tumor to grow lager than 2-3 mm in diameter, it must form a vasculature, a process known as angiogenesis. A selective p38 inhibitor has been shown to inhibit angiogenesis (see J. R. Jackson, J. Pharmacol Exp. Therpaeutics, 1998, 284, 687). Because angiogenesis is a critical component of the mass expansion of solid tumours, the development of new p38 kinase inhibitors for the inhibition of this process represents a promising approach for anti-tumour therapy. The compounds of the present invention are also useful in inhibiting growth of susceptible neoplasms (see R. M. Schultz, Potential of p38 MAP kinase inhibitors in the treatment of cancer. In: E. Jucker (editor), Progress in Drug Research 2003, 60, 59-92. The term “susceptible neoplasm” used in present application includes human cancers such as malignant melanoma, colorectal carcinoma, gastric carcinoma, breast carcinoma and non-small cell lung carcinoma.

Furthermore, inhibition of p38 kinase may be effective in treatment of certain viral conditions such as influenza (J. Immunology, 2000, 164, 3222), rhinovirus (J. Immunology, 2000, 165, 5211) and HIV (Proc. Nat. Acad. Sci., 1998, 95, 7422).

In summary, a number of inhibitors of p38 kinase are under active investigation for the treatment of a variety of disorders (Boehm, Adams Exp. Opin. Ther. Patents 2000, 10(1), 25-37. There remains a need for treatment in this field for compounds that are cytokine suppressive, i.e compounds that are capable of inhibiting p38 kinase.

### SUMMARY OF THE INVENTION

In a first embodiment the invention is a compound of formula (1)

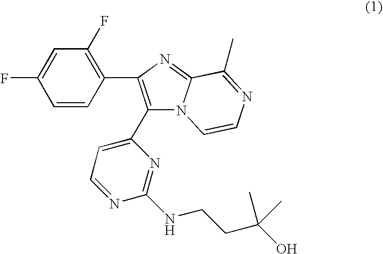

and pharmaceutically acceptable salts, prodrugs, and pharmaceutically active metabolites thereof.
In a second embodiment the invention is a compound of formula (2)

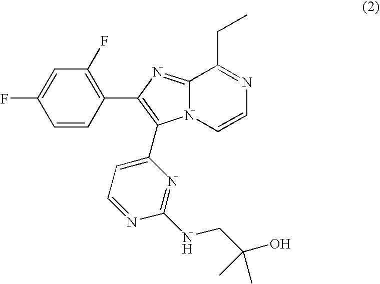

and pharmaceutically acceptable salts, prodrugs, and pharmaceutically active metabolites thereof.

In a third embodiment the invention is a compound of formula (3)

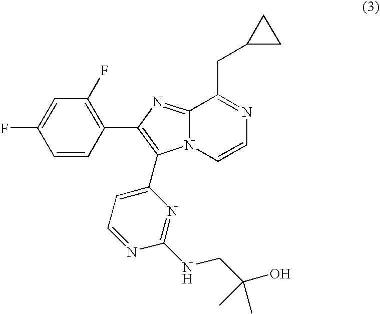

and pharmaceutically acceptable salts, prodrugs, and pharmaceutically active metabolites thereof.

### DETAILED DESCRIPTION OF THE INVENTION

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and Serine/Threonine kinases as a result of their substrate specificity. Serine/Threonine Kinases (S/T kinases) are a large sub-family of protein kinases that specifically transfer a phosphate group to a terminal hydroxyl moiety of specific serine or threonine residues (Hanks et al., (1988) Science, 241: 42-52). A number of S/T kinase family members are involved in inflammatory signaling, tumor growth or cellular transformation. For example, the mitogen-activated protein kinases (MAPKs) are S/T kinases that act as intermediates within the signaling cascades of Toll like receptors (TLRs), such as TLR4, growth/survival factors, such as EGF, and death receptors, such as the TNF receptor. Activation of MAPKs, such as extracellular signal-regulated kinases (ERK1-2), p38α, c-Jun N-terminal kinase (JNK) or MAPKAP-K2 (MK2) have been shown to transduce signaling in cells, such as monocytes/macrophages, resulting in the extracellular production of pro-inflammatory cytokines, such as TNF.

The p38 MAP kinase (p38, also known as CSBP or SAPK) signaling pathway has been reported to be responsible for the expression of pro-inflammatory cytokines (such as TNF, IL-1, IL-6, IL-8) that are elevated in many inflammatory and auto-immune diseases (see J. C. Lee, Nature Reviews Drug Discovery 2003, 2, 717-726 and references cited therein). This pathway has been shown to be activated by cellular stressors, such as osmotic shock, UV light, free radicals, bacterial toxins, viruses, cytokines, chemokines and in response, mediates the expression of several cytokines including, but not limited to, TNF, IL-1, IL-6 and IL-8. In cells of myeloid lineage, such as macrophages and monocytes, both IL-1 and TNFα are transcribed in response to p38 activation. Subsequent translation and secretion of these and other cytokines initiates a local or systemic inflammatory response in adjacent tissue and through infiltration of leukocytes. While this response is a normal part of the physiological response to cellular stress, acute or chronic cellular stress leads to the excess or unregulated expression of pro-inflammatory cytokines. This, in turn, leads to tissue damage, often resulting in pain and debilitation. (see G. Panayi, N Engl J Med 2001, 344(12), 907; J. Smolen Nature Reviews Drug Discovery 2003, 2, 473 and references cited therein). The four known isoforms of p38 MAP kinase (p38 α, β, γ, δ) each showing different expression levels, tissue distributions and regulation, support the concept that they are involved in the etiology of inflammatory, auto-immune and other diseases.

In summary, a number of inhibitors of p38 kinase are under active investigation for the treatment of a variety of disorders (Boehm, Adams Exp. Opin. Ther. Patents 2000, 10(1), 25-37). There remains a need for treatment in this field for compounds that are cytokine suppressive, i.e compounds that are capable of inhibiting p38 kinase.

Protein tyrosine kinases (PTKs) are enzymes that catalyse the phosphorylation of specific tyrosine residues in cellular proteins. This post-translational modification of these substrate proteins, often enzymes themselves, acts as a molecular switch regulating cell proliferation, activation or differentiation (for review, see Schlessinger and Ulrich, 1992, Neuron 9:383-391). Aberrant or excessive PTK activity has been observed in many disease states including benign and malignant proliferative disorders as well as diseases resulting from inappropriate activation of the immune system (e.g. autoimmune disorders), allograft rejection, and graft vs. host disease. In addition, endothelial-cell specific receptor PTKs such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, and infantile hemangiomas).

Tyrosine kinases can be of the receptor-type (having extracellular, transmembrane and intracellular domains) or the non-receptor type (being wholly intracellular).

Receptor Tyrosine Kinases (RTKs) comprise a large family of transmembrane receptors with diverse biological activities. At present, at least nineteen (19) distinct RTK subfamilies have been identified. The receptor tyrosine kinase (RTK) family includes receptors that are crucial for the growth and differentiation of a variety of cell types (Yarden and Ullrich, Ann. Rev. Biochem. 57:433-478, 1988; Ullrich and Schlessinger, Cell 61:243-254, 1990). The intrinsic function of RTKs is activated upon ligand binding, which results in phosphorylation of the receptor and multiple cellular substrates, and subsequently in a variety of cellular responses (Ullrich & Schlessinger, 1990, Cell 61:203-212). Thus, receptor tyrosine kinase mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response (e.g., cell division, differentiation, metabolic effects, and changes in the extracellular microenvironment; see Schlessinger and Ullrich, 1992, Neuron 9:1-20).

Non-receptor tyrosine kinases represent a collection of cellular enzymes which lack extracellular and transmembrane sequences. Over twenty-four individual non-receptor tyrosine kinases, comprising eleven (11) subfamilies (Src, Frk, Btk, Csk, Abl, Zap70, Fes/Fps, Fak, Jak, Ack and LIMK) have been identified. The Src subfamily of non-receptor tyrosine kinases is comprised of the largest number of PTKs and include Src, Yes, Fyn, Lyn, Lck, Blk, Hck, Fgr and Yrk. The Src subfamily of enzymes has been linked to oncogenesis and immune responses. A more detailed discussion of non-receptor tyrosine kinases is provided in Bohlen, 1993, Oncogene 8:2025-2031, which is incorporated herein by reference.

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

In a related aspect the invention provides a method for inhibiting p38 in a human subject suffering from a disorder in which p38 activity is detrimental, comprising administering to the human subject a compound of Formula 1, 2 or 3 such that p38 activity in the human subject is inhibited and treatment is achieved.

Many autoimmune diseases and disease associated with chronic inflammation, as well as acute responses, have been linked to activation of p38 MAP kinase and overexpression or dysregulation of inflammatory cytokines. The present compounds are useful in the treatment of inflammatory disorders including, but not limited to rheumatoid arthritis, osteoarthritis, asthma, chronic obstructive pulmonary disease (COPD), sepsis, psoriasis, psoriatic arthritis, inflammatory bowel disease, Crohn's disease, lupus, multiple sclerosis, juvenile chronic arthritis, Lyme arthritis, reactive arthritis, septic arthritis, spondyloarthropathy and systemic lupus erythematosus.

The compounds of the invention are also useful in the treatment of cardiovascular disorders, such as acute myocardial infarction, acute coronary syndrome, chronic heart failure, myocardial infarction, atherosclerosis, viral myocarditis, cardiac allograft rejection, and sepsis-associated cardiac dysfunction. Furthermore, the compounds of the present invention are also useful for the treatment of central nervous system disorders such as meningococcal meningitis, Alzheimer's disease and Parkinson's disease.

The compounds of the invention are also useful in the treatment of an ocular condition, a cancer, a solid tumor, a sarcoma, fibrosarcoma, osteoma, melanoma, retinoblastoma, a rhabdomyosarcoma, glioblastoma, neuroblastoma, teratocarcinoma, an cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, alpha-1 antitrypsin deficiency, amyotrophic lateral sclerosis, anemia, angina pectoris, anterior horn cell degeneration, anti cd3 therapy, antiphospholipid syndrome, anti-receptor hypersensitivity reactions, hypersensitivity reactions, hyperkinetic movement disorders, hypersensitivity pneumonitis, hypertension, hypokinetic movement disorders, aordic and peripheral aneurysms, hypothalamic-pituitary-adrenal axis evaluation, aortic dissection, arterial hypertension, arteriosclerosis, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphylaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, atrial fibrillation (sustained or paroxysmal), atrial flutter, atrioventricular block, B cell lymphoma, bone graft rejection, bone marrow transplant (BMT) rejection, small bowel transplant rejection, spinal ataxia, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic salicylate intoxication, colorectal carcinoma, congestive heart failure, conjunctivitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetic arteriosclerotic disease, Diffuses Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, gram negative sepsis, gram positive sepsis, granulomas due to intracellular organisms, hairy cell leukemia, Hallerrorden-Spatz disease, hay fever, heart transplant rejection, hemachromatosis, hemodialysis, hemolytic uremic syndrome/thrombolytic thrombocytopenic purpura, hemorrhage, idiopathic pulmonar fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile rheumatoid arthritis, juvenile spinal muscular atrophy, kidney transplant rejection, legionella, leishmaniasis, lipedema, liver transplant rejection, lymphederma, malaria, malignant Lymphoma, malignant histiocytosis, malignant melanoma, meningococcemia, metabolic/idiopathic, migraine headache, mitochondrial multi.system disorder, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, Kaposi's sarcoma, Hodgkin's disease, lymphoma, myeloma, leukaemia, malignant ascites, hematopoietic cancers Crow-Fukase (POEMS) syndrome (polyneuropathy, organomegaly, endocrinopathy, monoclonal gammopathy, and skin changes syndrome), a diabetic condition such as insulin-dependent diabetes mellitus glaucoma, diabetic retinopathy or microangiopathy, sickle cell anaemia, chronic inflammation, synovitis, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, Progressive supranucleo Palsy, primary pulmonary hypertension, radiation therapy, Raynaud's phenomenon and disease, Raynoud's disease, Refsum's disease, regular narrow QRS tachycardia, renovascular hypertension, restrictive cardiomyopathy, sarcoma, senile chorea, Senile Dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis A, Hepatitis B, Hepatitis C, His bundle arrythmias, HIV infection/HIV neuropathy, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, toxicity, transplants, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula 1, 2 or 3 of the invention can be used alone or in combination with another therapeutic agent to treat such diseases. It should be understood that the compounds of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the p38 inhibitors of this invention. Non-limiting examples of therapeutic agents for rheumatoid arthritis with which a compound of Formula 1, 2 or 3 of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. S/T kinase inhibitors of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, HUMIRA® (U.S. Pat. No. 6,090,382), CA2 (REMICADE™), CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula 1, 2 or 3 of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK or IKK), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of Formula 1, 2 or 3 of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. IRAK, NIK or IKK); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL4, IL-10, IL-11, IL-13 and TGFβ). Preferred examples of therapeutic agents for Crohn's disease with which a compound of Formula 1, 2 or 3 can be combined include the following: TNF antagonists, for example, anti-TNF antibodies, HUMIRA® (U.S. Pat. No. 6,090,382), CA2 (REMICADE™), CDP 571, TNFR-Ig constructs, (p75TNFRIgG (ENBREL™) and p55TNFRIgG (LENERCEPT™)) inhibitors and PDE4 inhibitors. A compound of Formula 1, 2 or 3 can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula 1, 2 or 3 can be combined include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula 1, 2 or 3 can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula 1, 2 or 3 may also be combined with agents such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of Formula 1, 2 or 3 can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula 1, 2 or 3 may also be combined with agents, such as alemtuzumab, dronabinol, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL4 agonists.

Non-limiting examples of therapeutic agents for angina with which a compound of Formula 1, 2 or 3 of the invention can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil HCl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril, bisoprolol fumarate, ibuprofen, diclofenac and misoprostol, naproxen, meloxicam, indomethacin, diclofenac, celecoxib, rofecoxib, sulfasalazine, methotrexate, azathioprine, minocyclin, prednisone, enteracept, infliximab, albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, tramcinolone acetonide, beclomethasone dipropionate, ipratropium bromide, azithromycin, pirbuterol acetate, theophylline anhydrous, methylprednisolone sodium succ, clarithromycin, zafirlukast, formoerol fumarate, influenza virus vaccine, methylprednisolone sodium succ, amoxicillin trihydrate, flunisolide/menthol, allergy injection, cromolyn sodium, fexofenadine hydrochloride, levofloxacin, inhaler assist device, guaifenesin, dexamthasone sodium phosphate, moxifloxacin HCl, doxycycline hyclate, fuaifenesin/d-methorphan, p-ephedrine/cod/chlorphenir, gatifloxacin, cetirizine hydrochloride, mometasone furoate, benzonatate, cephalexin, pe/hydrocone/chlorphenir, cetirizine HCl/pseucoephed, phenyphrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, metaproterenol sulfate, mesalamine, azathioprine, mercaptopurine, diphenoxylate/atrop sulf, loperamide hydrochloride, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, polycarbophil, propoxyphene napsylate, hydrocortisone, multivitamins, balsalazide disodium, codeine phosphate/apap, colesevelam HCl, cyanocobalamin, folic acid, levofloxacin, natalizumag, interferon-gamma, montelukast sodium, formoterol fumarate, triamcinolone acetonide, levofloxacin, guaifenesin, levalbuterol HCl, flunisolide, ceftriaxone sodium, gatifloxacin, amoxicillin/clavulanate, flunisolide/menthol, chlorpheniramine/hydrocodone metaproterenol sulfate, methylprednisolone, mometasone furoate, p-ephedrine/cod/chlorphenir, p-ephedrine/loratadine, terbutaline sulfate, tiotropium bromide, (R,R)-formoterol, TgAAT, Cilomilast, Roflumilast, Interferon-alpha-2α, Interferon-alpha-2β, Interferon-alpha con1, Interferon-alpha-n1, pegylated interferon-alpha-2a, Pegylated interferon-alpha-2β, Ribavirin, Peginterferon alfa-2β and ribavirin, Ursodeoxycholic Acid, Glycyrrhizic Acid, Thymalfasin, Maxamine, VX497, any compounds that are used to treat HCV through intervention with the following targets: HCV polymerase, HCV protease, HCV helicase, HCV IRES (internal ribosome entry site), azathioprine, colchicine, albuterol sulfate, gamma interferon, lorazepam, furosemide, lisinopril, cyclophosphamide, actinomycin d, alteplase, levofloxacin, metaproterenol sulfate, morphine sulfate, oxycodone HCl, triamcinolone acetonide, tacrolimus anhydrous, calcium, interferon-alpha, mycophenolate mofetil, Interferon-gamma-1b, clopidogrel bisulfate, atenolol, morphine sulfate, metoprolol succinate, warfarin sodium, isosorbide mononitrate, simvastatin, tenecteplase, torsemide, retavase, losartan potassium, quinapril HCl/mag carb, alteplase, enalaprilat, amiodarone hydrochloride, tirofiban HCl m-hydrate, diltiazem hydrochloride, captopril, irbesartan, propranolol hydrochloride, fosinopril sodium, lidocaine hydrochloride, eptifibatide, cefazolin sodium, atropine sulfate, aminocaproic acid, interferon, sotalol hydrochloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, cariporide, calcipotriene, clobetasol propionate, triamcinolone acetonide, halobetasol propionate, tazarotene, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, hydrocortisone, moisturizing formula, folic acid, desonide, coal tar, diflorasone diacetate, etanercept, lactic acid, methoxsalen, HCl/bismuth, subgal/znox/resor, methylprednisolone acetate, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, soap/tribromsalan, thimerosal/boric acid, cyclosporine, alefacept, efalizumab, pimecrolimus, PUVA, UVB, naproxen, leflunomide, hydroxychloroquine sulfate, prednisone, sulindac, betamethasone diprop augmented, triamcinolone acetonide, dimethylsulfoxide, piroxicam, diclofenac sodium, ketoprofen, nabumetone, tolmetin sodium, calcipotriene, cyclosporine, sodium/misoprostol, fluocinonide, glucosamine sulfate, gold sodium thiomalate, hydrocodone bitartrate/apap, risedronate sodium, sulfadiazine, thioguanine, valdecoxib, hydroxychloroquine sulfate, leflunomide, valdecoxib, methylprednisolone, azathioprine, triamcinolone acetonide, propoxyphene napsylate/apap, nabumetone, piroxicam, etodolac, oxaprozin, hydrocodone bitartrate/apap, fentanyl, human recombinant anakinra, tramadol HCl, salsalate, sulindac, yanocobalamin/fa/pyridoxine, acetaminophen, alendronate sodium, prednisolone, morphine sulfate, lidocaine hydrochloride, glucosamine sulf/chondroitin, cyclosporine, amitriptyline HCl, sulfadiazine, oxycodone HCl/acetaminophen, olopatadine HCl, misoprostol, omeprazole, mycophenolate mofetil, rituximab, IL-1 TRAP, MRA, CTLA4-IG, IL-18 BP, ABT-874, anti-IL18 antibody, Anti-IL15, BIRB-796, SCIO-469, X-702, AMG-548, VX-740, Roflumilast, IC-485, CDC-801, Mesopram, sirolimus, paclitaxel, everolimus, tacrolimus, ABT-578, hydrocodone bitartrate/apap, cyclobenzaprine HCl, oxycodone HCl/acetaminophen, Valdecoxib, codeine phosphate/apap, tramadol HCl/acetaminophen, metaxalone, methocarbamol, lidocaine hydrochloride diclofenac sodium, gabapentin, dexamethasone, carisoprodol, ketorolac tromethamine, diazepam, nabumetone, oxycodone HCl, tizanidine HCl, propoxyphene napsylate/apap, asa/oxycod/oxycodone ter, ibuprofen/hydrocodone bit, etodolac, propoxyphene HCl, amitriptyline HCl, carisoprodol/codeine phos/asa, morphine sulfate, orphenadrine citrate, temazepam, epidermal growth factor, corticosteroids, cyclosporin, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-1β monoclonal antibodies, anti-IL-6 monoclonal antibodies, growth factors, elastase inhibitors, pyridinyl-imidazole compounds, antibodies or agonists of TNF, LT, IL-1, IL-2, IL-6, IL-7, IL-8, IL-15, IL-16, IL-18, EMAP-II, GM-CSF, FGF, and PDGF, antibodies of CD2, CD3, CD4, CD8, CD25, CD28, CD30, CD40, CD45, CD69, CD90 or their ligands, cyclosporin, FK506, rapamycin, mycophenolate mofetil, leflunomide, NSAIDs, corticosteroids, prednisolone, phosphodiesterase inhibitors, adensosine agonists, antithrombotic agents, complement inhibitors, adrenergic agents, IRAK, NIK, IKK, IL-1β converting enzyme inhibitors, TNFα converting enzyme inhibitors, T-cell signalling inhibitors, metalloproteinase inhibitors, 6-mercaptopurines, angiotensin converting enzyme inhibitors, soluble cytokine receptors, soluble p55 TNF receptor, soluble p75 TNF receptor, sIL-1RI, sIL-1RII, sIL-6R, antiinflammatory cytokines, IL-4, IL-10, IL-11, IL-13 and TGFβ.

Non-limiting examples of therapeutic agents for ankylosing spondylitis with which a compound of Formula 1, 2 or 3 can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, etanercept, and infliximab.

Non-limiting examples of therapeutic agents for asthma with which a compound of Formula 1, 2 or 3 can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone and metaproterenol sulfate.

Non-limiting examples of therapeutic agents for COPD with which a compound of Formula 1, 2 or 3 can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula 1, 2 or 3 can be combined include the following: Interferon-alpha-2a, Interferon-alpha-2b, Interferon-alpha con1, Interferon-alpha-n1, pegylated interferon-alpha-2a, pegylated interferon-alpha-2b, ribavirin, peginterferon alfa-2b+ribavirin, ursodeoxycholic acid, glycyrrhizic acid, thymalfasin, Maxamine, VX497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula 1, 2 or 3 can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-1β.

Non-limiting examples of therapeutic agents for myocardial infarction with which a compound of Formula 1, 2 or 3 can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril HCl/mag carb, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula 1, 2 or 3 can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, and sulfasalazine.

Non-limiting examples of therapeutic agents for psoriatic arthritis with which a compound of Formula 1, 2 or 3 can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept and efalizumab.

Non-limiting examples of therapeutic agents for restenosis with which a compound of Formula 1, 2 or 3 can be combined include the following: sirolimus, paclitaxel, everolimus, tacrolimus, ABT-578, and acetaminophen.

Non-limiting examples of therapeutic agents for sciatica with which a compound of Formula 1, 2 or 3 can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Preferred examples of therapeutic agents for SLE (Lupus) with which a compound of Formula 1, 2 or 3 can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of Formula 1, 2 or 3 may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of Formula 1, 2 or 3 may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula 1, 2 or 3 can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula 1, 2 or 3 may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, HUMIRA® (U.S. Pat. No. 6,090,382), CA2 (REMICADE™), CDP 571, TNFR-Ig constructs, (p75TNFRIgG (ENBREL™) and p55TNFRIgG (LENERCEPT™)).

In this invention, the following definitions are applicable:

A “therapeutically effective amount” is an amount of a compound of Formula 1, 2 or 3 or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Physiologically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of Formula 1, 2 or 3 which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of Formula 1, 2 or 3 and their salts may exist in more than one crystal form and the present invention includes each crystal form and mixtures thereof.

Certain compounds of Formula 1, 2 or 3 and their salts may also exist in the form of solvates, for example hydrates, and the present invention includes each solvate and mixtures thereof.

Certain compounds of Formula 1, 2 or 3 may exist in different tautomeric forms or as different geometric isomers, and the present invention includes each tautomer and/or geometric isomer of compounds of Formula 1, 2 or 3 and mixtures thereof.

Certain compounds of Formula 1, 2 or 3 may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula 1, 2 or 3 and mixtures thereof.

Certain compounds of Formula 1, 2 or 3 may exist in zwitterionic form and the present invention includes each zwitterionic form of compounds of Formula 1, 2 or 3 and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —(CH₂)C(O)OH or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula 1, 2 or 3 wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl”, as used herein, include non-aromatic, ring systems, including, but not limited to, monocyclic, bicyclic and tricyclic rings, which can be completely saturated or which can contain one or more units of unsaturation, for the avoidance of doubt, the degree of unsaturation does not result in an aromatic ring system) and have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinesyl, piperazinyl, piperidinyl, pyrrolidinyl, quinicludinyl, thiomorpholinyl, tetrahydropyranyl and tetrahydrofuranyl.

The term “heteroaryl” as used herein, include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindole, benzo(b)thienyl, benzimidazolyl, benzofuranyl, benzoxazolyl, benzothiazolyl, benzothiadiazolyl, benzoxadiazolyl, furans, imidazoles, imidazopyridine, indole, indolinyl, indazoles, isoindolinyl, isoxazoles, isothiazoles, oxadiazoles, oxazoles, purine, pyrans, pyrazines, pyrazoles, pyridines, pyrimidines, pyrroles, pyrrolo[2,3-d]pyrimidine, pyrazolo[3,4-d]pyrimidine), quinolines, quinazolines, triazoles, thiazoles, thiophenyl, tetrahydroindole, tetrazoles, thiadiazoles, thienyls, thiomorpholines or tropanyl.

When the term “substituted heterocyclic” (or heterocyclyl) or “substituted heteroaryl” is used, what is meant is that the heterocyclic group is substituted with one or more substituents that can be made by one of ordinary skill in the art and results in a molecule that is a kinase inhibitor. For purposes of exemplification, which should not be construed as limiting the scope of this invention, preferred substituents for the heterocycle of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylcarbonyl, alkylester, alkyl-O—C(O)—, alkyl-heterocyclyl, alkyl-cycloalkyl, alkyl-nitrile, alkynyl, amido groups, amino, aminoalkyl, aminocarbonyl, carbonitrile, carbonylalkoxy, carboxamido, CF₃, CN, —C(O)OH, —C(O)H, —C(O)—C(CH₃)₃, —OH, —C(O)O-alkyl, —C(O)O-cycloalkyl, —C(O)O-heterocyclyl, —C(O)-alkyl, —C(O)-cycloalkyl, —C(O)-heterocyclyl, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, nitro, OCF₃, oxo, phenyl, —SO₂CH₃, —SO₂CR₃, tetrazolyl, thienylalkoxy, trifluoromethylcarbonylamino, trifluoromethylsulfonamido, heterocyclylalkoxy, heterocyclyl-S(O)ₚ, cycloalkyl-S(O)ₚ, alkyl-S—, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵-N(R)—S(O)₂Z²⁰⁰, -Z¹⁰⁵-N(R)—C(O)—N(R)-Z²⁰⁰, —N(R)—C(O)R, —N(R)—C(O)OR, OR—C(O)-heterocyclyl-OR, R_{c} and —CH₂OR_{c};
wherein R₃ is C₁-C₄ alkyl, C₃-C₆ cycloalkyl or phenyl;
wherein p is 0, 1 or 2;
where R_{c} for each occurrence is independently hydrogen, optionally substituted alkyl, optionally substituted aryl, —(C₁-C₆)—NR_{d}Rₑ, -E-(CH₂)ₜ—NR_{d}Rₑ, -E-(CH₂)ₜ—O-alkyl, -E-(CH₂)ₜ—S-alkyl, or -E-(CH₂)ₜ—OH;
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together to form a five- or six-membered heterocyclic ring.

An “heterocycloalkyl” group, as used herein, is a heterocyclic group that is linked to a compound by an aliphatic group having from one to about eight carbon atoms. For example, a preferred heterocycloalkyl group is a morpholinomethyl group.

As used herein, “aliphatic” or “an aliphatic group” or notations such as “(C₀-C₈)” include straight chained or branched hydrocarbons which are completely saturated or which contain one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons, which are completely saturated. Preferred alkyls are methyl, ethyl, propyl, butyl, pentyl, hexyl and isomers thereof. As used herein, “alkenyl” and “alkynyl” means C₂-C₈ and includes straight chained or branched hydrocarbons which contain one or more units of unsaturation, one or more double bonds for alkenyl and one or more triple bonds for alkynyl.

As used herein, aromatic groups (or aryl groups) include aromatic carbocyclic ring systems (e.g. phenyl and cyclopentyldienyl) and fused polycyclic aromatic ring systems (e.g. naphthyl, biphenylenyl and 1,2,3,4-tetrahydronaphthyl).

As used herein, cycloalkyl means C₃-C₁₂ monocyclic or multicyclic (e.g., bicyclic, tricyclic, etc.) hydrocarbons that is completely saturated or has one or more unsaturated bonds but does not amount to an aromatic group. Preferred examples of a cycloalkyl group are cyclopropyl, cyclobutyl, cyclopentyl, cyclopentenyl, cyclohexyl and cyclohexenyl.

As used herein, amido group means —NHC(═O)—.

As used herein, acyloxy groups are —OC(O)R.

As used herein, many moieties or substituents are termed as being either “substituted” or “optionally substituted”. When a moiety is modified by one of these terms, unless otherwise noted, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinyl-alkoxy, alkyl groups (which itself can also be substituted, such as —C₁-C₆-alkyl-OR, —C₁-C₆-alkyl-N(R)₂, and —CF₃), alkylamino, alkylcarbonyl, alkylester, alkylnitrile, alkylsulfonyl, amino, aminoalkoxy, CF₃, COH, COOH, CN, cycloalkyl, dialkylamino, dialkylaminoalkoxy, dialkylaminocarbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, nitro, oxo, OCF₃, optionally substituted phenyl, S(O)₂CH₃, S(O)₂CF₃, and sulfonyl, N-alkylamino or N,N-dialkylamino (in which the alkyl groups can also be substituted).

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of Formula 1, 2 or 3 as a medicament.

A further aspect of the present invention provides the use of a compound of Formula 1, 2 or 3 or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of Formula 1, 2 or 3 to a mammal, particularly a human being, in need thereof.

### Enzyme Assays

The in vitro potency of compounds of Formula 1, 2 or 3 in inhibiting one or more of the protein kinases discussed herein or described in the art may be determined by the procedures detailed below.

The potency of compounds of Formula 1, 2 or 3 can be determined by the amount of inhibition of the phosphorylation of an exogenous substrate (e.g., a synthetic peptide (Z. Songyang et al., Nature. 373:536-539) by a test compound relative to control.

### p38 Kinase Assay

Materials: Active p38α enzyme can be purchased from Upstate Biotechnology Inc. (UBI). Anti-phospho-MBP specific antibody can be purchased from UBI and Europium (Eu)-cryptate labeled by Cis-Bio International. SAXL (streptavidine linked XL) can be obtained for Prozyme. Biotin-MBP-peptide (Biot-Ahx-VHFFKNIVTPRTPPPSQGKGAEGQR-OH) can be made by New England Peptide. HTRF reader RUBYstar was can be acquired from BMG Labtech.
The kinase assay is performed using the homogenous time-resolved fluorescence (HTRF) method (Mabile, 1991; Mathis, 1993). The assay mixture contains 7.8 nM p38α, 0.5 μM biotin-MBP-peptide, 0.1 mM ATP and compound (to a final 5% DMSO) in a buffer containing 20 mM MOPS pH 7.2, 10 mM MgC₂, 5 mM EGTA, 5 mM β-phosphoglycerol, 1 mM Na₃VO₄, 0.01% Triton-X-100, 1 mM DTT. The reaction is carried out at room temperature in 96 half-well black plates (Corning). At designated time point, EDTA (to a final 0.1 M) is added to quench the reaction. The products are detected by addition of the revelation reagents (to a final 11 ng anti-phospho-MBP-Eu antibody and 0.34 μg SAXL). The plates are incubated in dark at 4° C. overnight, and read in the HTRF reader RUBYstar. The ratio between the signal at 620 nm and 665 nm at various inhibitor concentrations is used to calculate the IC₅₀.

### REFERENCE

(1) M. Mabile, G. Mathis, E. J. P., Jolu, D. Pouyat, C. Dumont, Patent WO 92:13264, 1991
(2) G. Mathis, Clin. Chem. 39 (1993) 1953-1959

### Methods

Kinase assays: The kinase assays were performed using the homogenous time-resolved fluorescence (HTRF) method (Mabile, et al.; Mathis, et al.). IKKα and IKKβ (made in house) assay contained either 6.7 nM IKKα or 1.7 nM IKKβ, 0.5 μM biotin-IκBα-peptide (Cell Signaling), 0.01 mM ATP and compound in IKK buffer (20 mM MOPS pH 7, 10 mM MgCl₂, 5 mM EGTA, 5 mM β-phosphoglycerol, 1 mM Na₃VO₄, 0.01% Triton-X-100, 1 mM DTT, 5% DMSO). p38α and CDK2 (UBI) assays contained either 7.8 nM p38α or 2.7 nM CDK2/cyclin A, and 0.5 μM biotin-MBP-peptide, 0.1 mM ATP and compound in the IKK Buffer. p38β assay contained 0.3 nM p38β, and 0.1 μM biotin-MBP-protein (UBI), 0.1 mM ATP and compound in the IKK Buffer. JNK1, JNK2 and JNK3 assays contained either 11.1 nM JNK1, 7.6 nM JNK2, or 2.4 nM JNK3, 1 μM biotin-ATF2-peptide (Cell Signaling), 0.01 mM ATP and compound in the IKK Buffer. KDR (make in house) assay contained 4.0 nM KDR, 2 μM biotin-FGFR-peptide, 0.1 mM ATP and compound in a buffer containing 50 mM HEPES, pH 7.1, 10 mM MgCl₂, 2 mM MnCl₂, 2.5 mM DTT, 0.01% BSA, 0.1 mM Na₃VO₄ and 5% DMSO. JAK1 (make in house) assay contained 3.6 nM JAK1, 2 μM biotin-FGFR-peptide, 0.001 mM ATP and compound in a buffer containing 50 mM MOPSO, pH 6.5, 10 mM MgCl₂, 2 mM MnCl₂, 2.5 mM DTT, 0.01% BSA, 0.1 mM Na₃VO₄ and 5% DMSO. All assays were carried out at RT for 60 min and stopped by addition of EDTA. The products were detected by addition of revelation reagents containing Europium labeled phospho-specific antibodies and SAXL. The plates were incubated in dark at 4° C. overnight, and read in the HTRF reader RUBYstar (BMG).

### REFERENCE

(3) M. Mabile, G. Mathis, E. J. P., Jolu, D. Pouyat, C. Dumont, Patent WO 92/13264, 1991
(4) G. Mathis, Clin. Chem. 39 (1993) 1953-1959

### Cellular Assays

THP-1 cells from ATCC (TIB-202) are serum-starved and seeded at a density of 2×10⁵/well in 100 μL of low serum RPMI media (0.5% FBS). 50 μl samples of compounds in appropriate serial dilutions are added to the wells. Compound stocks and dilutions in 100% DMSO are prepared such that final concentration of DMSO in RPMI media is 0.5%. Cells and compounds or controls are pre-incubated for 1 hour in a 37° C. incubator.

Cytokine release and P-Hsp27 induction is stimulated by LPS treatment. LPS (Sigma, L-4516) is reconstituted to a concentration of 1 mg/ml in endotoxin free dIH₂O, diluted in RPMI media such that 50 μl/well is added to each well for a final concentration of 1 μg/ml (excepting negative control wells). Plates with cells, compound and LPS are incubated at 37° C. for 45 minutes. This time point needs recalibration when new THP-1 cells are thawed.

For analysis of P-Hsp27 (phosphorylated Hsp27 protein), plates are vacuum filtered to remove media and compounds. Cells are washed twice with buffer (UBI, Assay Buffer #1, 43-010) using vacuum filtration. Then, 100 μl of cell lysis buffer (Biorad, 171-304011) is added per well and the plate is covered and shaken for 20 mins at 4° C. to lyse cells. Lysates are directly transferred to a flat bottom 96 well plate for analysis or stored frozen at −20° C. until analysis. Lysates are diluted 1:2 with assay buffer #1 and analysed by the Luminex method on a Bio-Plex machine following manufacturers directions (UBI, Phospho-HSP27 Beadmates kit, 46-607).

For analysis of cytokine release, plates are spun after incubation with LPS for 5 min at 1000 rpm and 100 μl of supernatant media is directly transferred to a 2^{nd} 96 well plate. Test plate with cells is returned to incubator O/N to be assayed for toxicity the next day (see below). Supernatant is stored at −20° C. until analysis. Supernatant media sample plates are analyzed in a standard ELISA format following manufacturers instructions (R&D, huTNFα ELISA assay kit). Toxicity analysis is done after the overnight incubation with compound. 50 μl of a 2.5 mg/ml solution of MTT (Sigma, M 2128) is added to cells. Plate is incubated at 37° C. for 3 hrs. 50 μl of 20% SDS is then added to solubilize the formazen dye. Plates are incubated at 37° C. for an additional 3 hrs and OD570 is measured on a spectrophotometer.

### Materials:

Blood donors are in-house volunteers. Tubes used for drawing blood are 3.2% Buffered Sodium Citrate from Monoject, Mansfield, Mass., Catalog Number 340486. Dilution Plates and Assay Plates were from Corning, COSTAR Catalogs Numbers 3365 and 3599, respectively. Dimethyl sulphoxide (DMSO) was from Sigma, St. Louis, Mo., Catalog Number D2650. RPMI Media 1640 and HEPES Buffer Solution (1M) are from Invitrogen GIBCO Cell Culture Systems, Carlsbad, Calif., Catalog Numbers 11875 and 15630. Lipopolysaccharides from Escherichia coli 0127:B8 (LPS) was from Sigma, Catalog Number L4516. Tumor Necrosis Factor Alpha (TNF-α/TNFSF1A) ELISA kits were from R&D Systems, Inc., Minneapolis, Minn., Catalog Number PDTA00C.

### Methods:

Blood is drawn from healthy donors into sodium citrate tubes within 1 hour of assay. Drugs were prepared in Dimethyl sulphoxide (DMSO) and serial dilute (1:3) with DMSO in Dilution Plate(s) to give 8 dilution points for each compound tested. Further dilution (1:100) of drug was made into RPMI Media 1640, 20 mM HEPES. Into wells of 96-well Assay Plate(s), 100 μl/well of diluted drug or control (1% DMSO in RPMI Media 1640, 20 mM HEPES) and 80 μL of blood is applied and pre-incubated for 30 minutes in an incubator set at 37 degrees centigrade. Tumor Necrosis Factor Alpha (TNF-α) is then stimulated with the addition of Lipopolysaccharides from Escherichia coli 0127:B8 (LPS, 50 ng/ml) for 3.5 hours at 37 degrees centigrade. Plates are spun at 183 g (1000 rpm in Beckman/Coulter Allegra 6KR centrifuge) for 10 minutes. Cell-free supernatant (75 μL/well) was collected and TNF-α is measured by commercial ELISA kit, following protocol of manufacturer. Potency of drug to inhibit TNF-α in vitro is determined the percent reduction of measured TNF-α in wells with drug compared to control wells without drug. Results are represented as IC₅₀ values.

### Reference: Current Protocols in Immunology (2005) 7.18B-7.18B12.

### LPS-Induced TNF Production In Vivo

### Materials:

Lipopolysaccharide (LPS) from Escherichia coli, serotype 0111:B4 (Sigma, cat #L-4130, lot #095K4056)

### Phosphate Buffered Saline pH 7.2 (Gibco)

### PEG 200 (Sigma, cat #P3015)

### Methylcellulose (Sigma, cat #M7027)

Male Lewis rats, 200-300 g (Charles River Laboratories)

### Rat Tumor Necrosis Factor α (TNFα) ELISA kit (R&D Systems cat #RTA00)

### Methods:

The test compound is prepared into vehicle (5% PEG 200, in 0.5% Methylcellulose) at the desired concentrations for dosing (1, 3, 10, 30, 100 mg/kg). Lewis rats are pre-dosed with the compound(s) either intraperitoneally (i.p.) or orally (p.o.) at 0.002 ml/gram body weight one-two hours prior to the LPS challenge. Negative control includes rats treated with vehicle (5% PEG 200, in 0.5% Methylcellulose) alone. LPS is dissolved in phosphate buffered saline, sonicated and the rats are injected with 1 mg/kg intravenously (i.v.) at 0.001 ml/gram body weight. One hour after the LPS challenge the rats are cardiac bled and the serum is analyzed for TNFα by ELISA. The compound concentration is also determined in the serum.

The average concentration of TNFα in the vehicle treated group is taken as a maximal (100 percent) response. The mean TNFα levels in the compound treated groups are expressed as a percent of the maximal response. The percent of maximal TNFα responses at various doses or serum concentrations of the compound(s) are further analyzed using a four parameter curve fit of logarithmically transformed data (Graphpad Prism 4 software) to generate ED₅₀ and EC₅₀.

### Relevant Reference(s):

Azab A, et al. (1998). Life Sci. 63: 323-327.
Martinez E F, et. al (2004) Biochem. Pharma. 68:1321-1329.

The teachings of all references, including journal articles, patents and published patent applications, are incorporated herein by reference in their entirety.

Compounds of the invention may be prepared using the synthetic scheme illustrated in Scheme 1. Starting materials are commercially available or may be prepared by the procedures described herein or by procedures that would be well known to one skilled in the art of organic chemistry. The variables used in the Scheme are as defined herein or as in the claims.

A method for preparing imidazopyrazine compounds of the invention is illustrated in Scheme 1. In Scheme 1, step i, a suitably substituted α-bromoketone 1 is reacted with an optionally substituted 2-amino heterocycle 2. These types of cyclization reactions are well established in the literature (see, for example, Spitzer, et al., J Med Chem 1988, 31, 1590-1595). This reaction is typically conducted in an organic solvent (such as ACN, EtOH or DMF) at temperatures at or below reflux (such as 80° C.). The product 3 is typically isolated from the reaction mixture as a solid by concentrating the mixture and then is used crude after extractive work up with a suitable organic solvent (such as IPA, DCM or EtOAc) or is purified either by crystallizing or triturating in an organic solvent (such as DCM, EtOH or EtOAc) or by flash silica gel chromatography. Compounds 3 can be used as is or first undergo functional group manipulation using methods known to one skilled in the art (see, for example, Larock, R. C. Comprehensive Organic Transformations: A Guide to Functional Group Preparations, 2^{nd} edition, 1999, Wiley-VCH Publishers, New York). In a non-limitating example where R1=Cl, an alkyl group may be introduced via an iron-mediated addition of a Grignard reagent using conditions such as those described in Furstner et al., J Am Chem Soc, 2002, 124, 13856-13863. In an alternate, non-limiting, example where R1=Cl, an alkyl group is introduced by reaction with an alkylidenephosphorane (see, for example, Taylor, E. C. and Martin, S. F, J Am Chem Soc, 1974, 96, 8095-8102. Coupling of compounds 3 with a substituted pyrimidine such as heterocycles 4 to produce compounds 5 as shown in step ii (Scheme 1) is frequently conducted with palladium-mediated arylation using a catalyst/ligand system such as Pd(OAc)₂/PPh₃ or PdCl₂(PPh₃)₂ (see, for example, Pivsa-Art, et al., Bull Chem Soc Japan, 1998, 71, 467-473). This reaction is typically carried out with a base (such as Cs₂CO₃, CsOAc, or KOAc) at elevated temperatures (for example, 80-100° C.) in a solvent such as DMF or NMP. Oxidation as shown in step iii (Scheme 1) is typically accomplished by treating a solution of 5 in an organic solvent (such as DCM and/or MeOH) with an oxidant (such as an aqueous solution of Oxone® or m-CBPA) at room temperature to produce 6 (see, for example, Kennedy, R. J. and Stock, A. M. J Org Chem, 1960, 25, 1901-1906 or Zanatta, et al., Synthesis 2003, (6), 894-898). Displacement of the sulfone leaving group of 6 with a primary amine to provide 7 as shown in step iv (Scheme 1) can be accomplished by a variety of methods known to one skilled in the art. For example, compounds 6 are reacted with the desired primary amine in an organic solvent (such as dioxane, toluene, or DMSO), with or without a hindered organic base (such as TEA), at elevated temperatures (see, for example, Clark, et al., J Med Chem, 2004, 47, 2724-2727). The compounds 7 can then be isolated and purified using standard techniques (such as crystallization, flash column chromatography, or reverse-phase liquid chromatography).

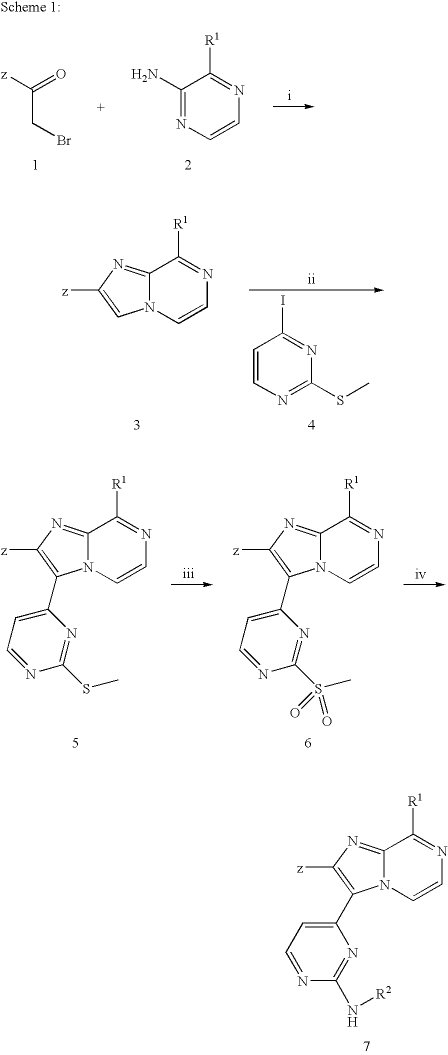

### Abbreviations

### ACN Acetonitrile

bp Boiling point
CsOAc Cesium acetate
DCM Dichloromethane (methylene chloride)

### DME 1,2-Dimethoxyethane

### DMF N,N-Dimethylformamide

EtMgBr Ethyl magnesium bromide
EtOAc Ethyl acetate
Et₂O Diethyl ether
IPA Isopropyl alcohol
KOAc Potassium acetate
MeMgBr Methyl magnesium bromide
MeOH Methyl alcohol

### NMP N-methylpyrrolidinone

Pd(OAc)₂ Palladium(II) acetate

### PPh₃ Triphenylphosphine

### THF Tetrahydrofuran

TLC Thin layer chromatography

TABLE 1
LC/MS methods

| Method | Conditions |
|---|---|
| a | 5% to 95% ACN/0.01M aqueous ammonium acetate over |
| | 3.7 min with a hold at 95% ACN/0.01M aqueous ammonium |
| | acetate for 1 min at 1.3 mL/min; Zorbax XDB C18, |
| | 5 μm, 50 × 4.6 mm column. Detection methods are |
| | diode array (DAD) and evaporative light scattering |
| | (ELSD) detection as well as positive/negative |
| | electrospray ionization. |
| b | 5% to 95% ACN/0.01M aqueous ammonium acetate over |
| | 2.0 min; 95% ACN/0.01M aqueous ammonium acetate for |
| | 1.5 min at 1.4 mL/min; UV λ = 210-360 nm; |
| | Genesis C8, 4 μm, 30 × 4.6 mm column. Detection |
| | methods are diode array (DAD) and evaporative light |
| | scattering (ELSD) detection as well as positive/ |
| | negative electrospray ionization. |

### EXAMPLES

### Example #1

### 4-{4-[2-(2,4-Difluorophenyl)-8-methylimidazo[1,2-a]pyrazin-3-yl]-pyrimidin-2-ylamino}-2-methyl-butan-2-ol

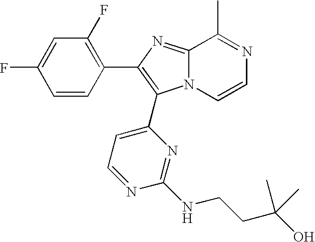

### Step A

### 8-Chloro-2-(2,4-difluorophenyl)-imidazo[1,2-α]pyrazine

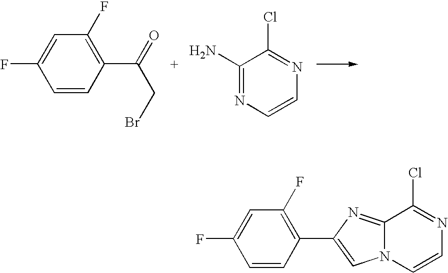

A mixture of 2-bromo-1-(2,4-difluorophenyl)ethanone (107.31 g, 442.89 mmol) and 3-chloropyrazin-2-amine (98.00 g, 756.5 mmol) in ACN (800 mL) was stirred at reflux for about 20 h. The reaction mixture was cooled to about 25° C. before the resultant solid was collected. The filtrate solvent was removed in vacuo to yield a brown solid. This filtration was done to negate the bumping associated with the removal of the ACN. The combined solids were then suspended in water (750 mL) and basified, whilst stirring, with 2N NaOH (750 mL). After about 30 min, the product was partitioned between DCM (9×1000 mL) and filtered from the insoluble material to aid extraction process. The organic extracts were combined and stirred with 2.5N HCl (4×750 mL). The organic layer was finally washed with 2.0N NaOH (500 mL) and water (2×500 mL), dried over MgSO₄, and filtered through a Florisil® pad (3 inch diameter×3 inch depth) to remove origin material. The Florisil® pad was washed with repeated amounts of solvent until no product was detected by TLC. The organic solvent was removed in vacuo to yield a yellow solid. The solid was suspended in IPA (200 mL) at about 80° C. for about 15 min and then cooled to about 20° C. The solid was collected and washed with ice-cold IPA (2×40 mL), followed by petroleum ether [bp 30-60° C.] (3×80 mL) to remove impurities. The solid was dried in vacuo at about 70° C. overnight to yield the title compound as a yellow powdery solid (69.75 g, 57%): LC/MS (Table 1, Method a) Rₜ=2.70 min; MS m/z: 266.1 (M+H)⁺.

### Step B

### 2-(2,4-Difluorophenyl)-8-methylimidazo[1,2-α]pyrazine

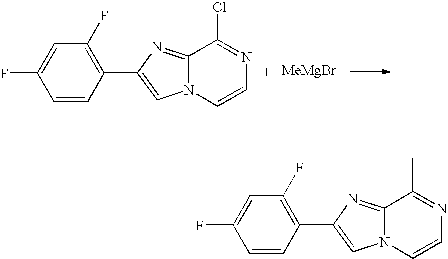

Into a 3-neck reaction flask equipped with a mechanical stirrer, was added 8-chloro-2-(2,4-difluorophenyl)imidazo[1,2-a]pyrazine (40.00 g, 150.6 mmol), ferric acetylacetonate (2.66 g, 7.53 mmol), THF (970 mL) and NMP (86 mL). The flask was charged with nitrogen and cooled to about −5-0° C. before the drop-wise addition of 3M MeMgBr in Et₂O (150 mL) over about 20 min. After the addition was complete, the reaction was stirred for about 15 min before the cooling bath was removed. The reaction mixture was allowed to warm to ambient temperature over about 1 hour and then stirred overnight. The reaction was concentrated and the residue was stirred with water (1000 mL) and EtOAc (1000 mL) for about 15 min. The mixture was filtered through a Celite® pad to remove salts. The Celite® pad was scraped and stirred with EtOAc (3×250 mL). The basic aqueous phase was separated and extracted with EtOAc (3×250 mL). The organic layers were combined, washed with water (3×350 mL), dried over MgSO₄, and filtered through a Florisil® pad to remove origin material. The solvent was removed in vacuo to yield a yellow solid that was treated with boiling MeOH (125 mL), cooled to about 15° C. and treated with petroleum ether [bp 30-60° C.] (250 mL) while stirring. The resulting solid was filtered, washed with petroleum ether [bp 30-60° C.] (3×50 mL), and dried in vacuo at 70° C. to yield the title compound as a pale yellow powdery solid (23.25 g, 64%): LC/MS (Table 1, Method a) Rₜ=2.42 min; MS m/z: 246.1 (M+H)⁺.

### Step C

### 2-(2,4-Difluorophenyl)-8-methyl-3-(2-methylsulfanylpyrimidin-4-yl)-imidazo[1,2-α]pyrazine

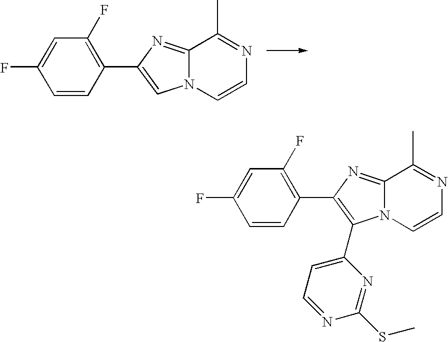

A mixture of PPh₃ (4.28 g, 16.3 mmol) and Pd(OAc)₂ (1.83 g, 8.16 mmol) were stirred in DMF (90 mL). The mixture was degassed with nitrogen then heated at about 100° C. for about 10 min until it was a dark red solution. The reaction was removed from heating then 2-(2,4-difluorophenyl)-8-methylimidazo[1,2-a]pyrazine (10.0 g, 40.8 mmol) and CsOAc (15.7 g, 81.8 mmol) were added. The reaction was returned to heating at about 100° C. and a solution of 4-iodo-2-(methylthio)pyrimidine (20.56 g, 81.6 mmol) in DMF (40 mL) was added via addition funnel over about 4 h. The reaction was heated at about 100° C. for about 16 hours after the addition ended then cooled to ambient temperature and concentrated under reduced pressure. The resulting solid was dissolved in DCM (500 mL) and washed with 1 N HCl (5×200 mL) then washed with 0.5 N NaOH (200 mL) and filtered through Celite® to break the resulting emulsion. The layers were separated and the organic layer was dried over MgSO₄, filtered, and concentrated under reduced pressure. The resulting residue was filtered through a silica gel plug using EtOAc then concentrated under reduced pressure and dissolved in DCM (200 mL). The DCM solution was purified by silica gel chromatography (330 g column) using a gradient of DCM:ACN (1:0 for 4 min, cut to 4:1 and held for 40 min, ramped to 1:1 over 20 min and held until product finished eluting). All product-containing fractions (including those with triphenylphosphine oxide) were combined and concentrated under reduced pressure until heavy precipitate present in a dark liquid. Then IPA was added and further concentrated under reduced pressure to remove DCM. The resulting suspension was filtered, washing with IPA followed by petroleum ether (b.p. 30-60° C.) and dried in vacuum oven at about 70° C. to give the title compound (5.7 g, 38%): LC/MS (Table 1, Method a) Rₜ=3.39 min; MS m/z: 370.3 (M+H)⁺.

### Step D

### 2-(2,4-Difluorophenyl)-3-(2-methanesulfonylpyrimidin-4-yl)-8-methylimidazo[1,2-α]pyrazine

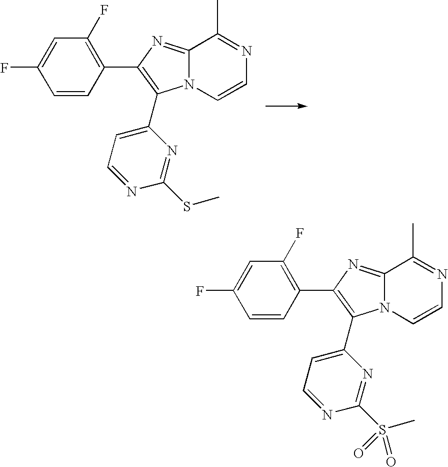

To a rapidly stirred solution of 2-(2,4-difluorophenyl)-8-methyl-3-(2-methylsulfanylpyrimidin-4-yl)-imidazo[1,2-α]pyrazine (17.2 g, 46.6 mmol) in DCM (373 mL) and MeOH (373 mL) was added a solution of Oxone® (57.4 g, 93.4 mmol) in water (187 mL) at ambient temperature. After about 16 hours, the mixture was diluted with water (900 mL) and extracted with DCM (3×300 mL). The combined organic layers were washed with brine (300 mL), dried over MgSO₄, filtered, and concentrated under reduced pressure. The crude material was purified by silica gel chromatography (330 g column) using a gradient of DCM:EtOAc (1:1 for 10 min, ramped to 0:1 over 20 min and held for additional 20 min). Fractions enriched in product were crystallized from hot ACN. The resulting solid was filtered, washing with additional ACN, and dried in a vacuum oven at about 70° C. for about 5 hours to give 15.9 g (85%) of the title compound. Additional product can be obtained from further purification of filtrate: LC/MS (Table 1, Method b) Rₜ=1.77 min; MS m/z: 402.1 (M+H)⁺.

### Step E

### 4-{4-[2-(2,4-Difluorophenyl)-8-methylimidazo[1,2-a]pyrazin-3-yl]-pyrimidin-2-ylamino}-2-methyl-butan-2-ol

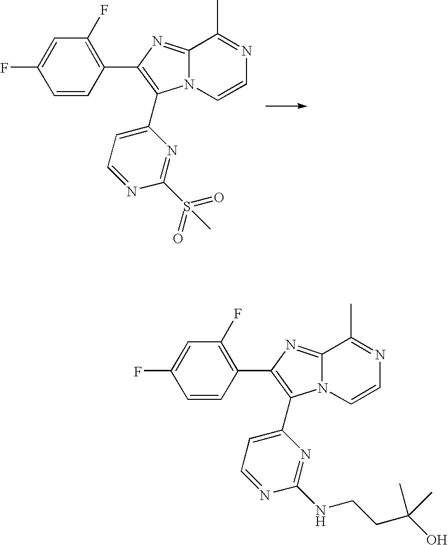

To a mixture of 2-(2,4-difluorophenyl)-3-(2-methanesulfonylpyrimidin-4-yl)-8-methyl-imidazo[1,2-a]pyrazine (20.0 g, 49.8 mmol) in ACN (200 mL) was added 4-amino-2-methylbutan-2-ol (WO 2003101968 A1, 19.5 g, 189 mmol). The mixture was heated at about 80° C. overnight. After cooling to ambient temperature, the reaction mixture was filtered and the solid was re-crystallized from hot ACN three times sequentially. The resulting solid was dried at about 70° C. under vacuum to yield the title compound (14.8 g, 70%): LC/MS (Table 1, Method a) Rₜ=2.93 min; MS m/z: 425.4 (M+H)⁺, mp 175° C.

### Example #2

### 1-{4-[2-(2,4-Difluorophenyl)-8-ethylimidazo[1,2-a]pyrazin-3-yl]-pyrimidin-2-ylamino}-2-methylpropan-2-ol

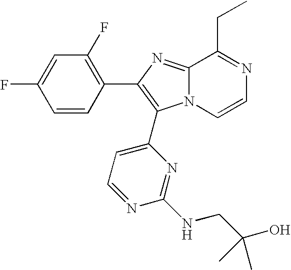

### Step A

### 8-Chloro-2-(2,4-difluorophenyl)-imidazo[1,2-α]pyrazine

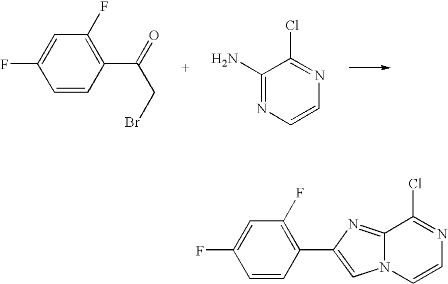

A mixture of 2-bromo-1-(2,4-difluorophenyl)ethanone (107.31 g, 442.89 mmol) and 3-chloropyrazin-2-amine (98.00 g, 756.5 mmol) in ACN (800 mL) was stirred at reflux for about 20 hours. The reaction mixture was cooled to about 25° C. before the resultant solid was collected. The filtrate solvent was removed in vacuo to yield a brown solid. This filtration was done to negate the bumping associated with the removal of the ACN. The combined solids were then suspended in water (750 mL) and basified, whilst stirring, with 2N NaOH (750 mL). After about 30 min, the product was partitioned between DCM (9×1000 mL) and filtered from the insoluble material to aid extraction process. The organic extracts were combined and stirred with 2.5N HCl (4×750 mL). The organic layer was finally washed with 2.0N NaOH (500 mL) and water (2×500 mL), dried over MgSO₄, and filtered through a Florisil® pad (3 inch diameter×3 inch depth) to remove origin material. The Florisil® pad was washed with repeated amounts of solvent until no product was detected by TLC. The organic solvent was removed in vacuo to yield a yellow solid. The solid was suspended in IPA (200 mL) at about 80° C. for about 15 min and then cooled to about 20° C. The solid was collected and washed with ice-cold IPA (2×40 mL), followed by petroleum ether [bp 30-60° C.] (3×80 mL) to remove impurities. The solid was dried in vacuo at about 70° C. overnight to yield the title compound as a yellow powdery solid (69.75 g, 57%): LC/MS (Table 1, Method a) Rₜ=2.70 min; MS m/z: 266.1 (M+H)⁺.

### Step B

### 2-(2,4-Difluorophenyl)-8-ethylimidazo[1,2-α]pyrazine

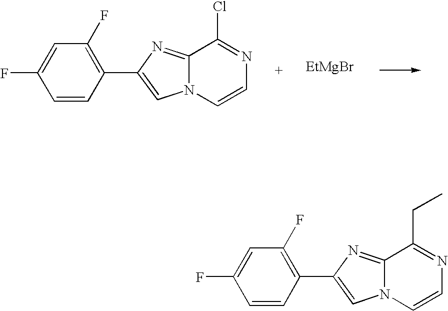

A solution of bromoethane (42.1 mL, 565 mmol) in anhydrous Et₂O (50 mL) was added dropwise to a stirred suspension of magnesium turnings (13.7 g, 565 mmol) in Et₂O (150 mL) over about 1 hour, maintaining the internal temperature to about 25-30° C. The reaction mixture was then stirred at ambient temperature overnight. In a separate flask, a suspension of 8-chloro-2-(2,4-difluorophenyl)imidazo[1,2-a]pyrazine (50.0 g, 188 mmol), ferric acetylacetonate (3.32 g, 9.41 mmol), NMP (107 mL, 1111 mmol) and THF (1000 mL) was stirred at about −5-0° C. for about 5 min before the dropwise addition of the freshly prepared solution of EtMgBr in Et₂O (from above) was added dropwise over about 1 h. After the addition was complete, the reaction was stirred at about −5-0° C. for about 30 min before the cooling bath was removed and the reaction was allowed to warm to ambient temperature overnight. The solvent was removed in vacuo and the residue was diluted with water (1000 mL) and EtOAc (1000 mL) then stirred for about 15 min and filtered through a Celite® pad. The brown paste (product and Mg salts) collected was removed from the pad and stirred with EtOAc (300 mL); this procedure was repeated two times. The original aqueous layer was extracted with these washings. The combined organic layers were washed with water (4×500 mL), dried over MgSO₄, filtered, and concentrated under reduced pressure to yield a brown solid. The solid was triturated with boiling MeOH (60 mL) then cooled to about 15-20° C. and the solid was collected, washed with ice-cold MeOH (2×20 mL) followed by petroleum ether [bp 30-60° C.] (3×25 mL) and dried to yield a dark fawn powdery solid (29.4 g, 58%): LC/MS (Table 1, Method b) Rₜ=2.20 min; MS m/z: 260.1 (M+H)⁺.

### Step C

### 2-(2,4-Difluorophenyl)-8-ethyl-3-(2-methylsulfanylpyrimidin-4-yl)-imidazo[1,2-α]pyrazine

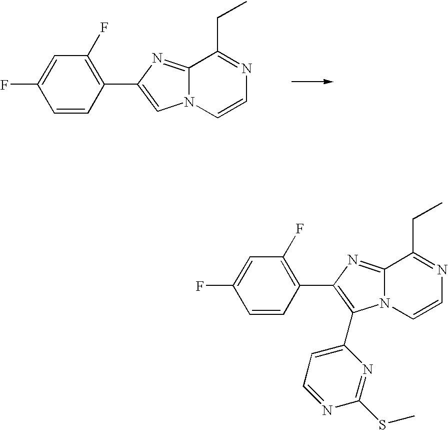

A mixture of PPh₃ (6.12 g, 23.3 mmol) and Pd(OAc)₂ (2.62 g, 11.7 mmol) were stirred in DMF (136 mL). The mixture was degassed with nitrogen then heated at about 100° C. for about 15 min until it was a dark red solution. The reaction was removed from heating then 2-(2,4-difluorophenyl)-8-ethylimidazo[1,2-a]pyrazine (15.1 g, 58.3 mmol) and CsOAc (15.7 g, 81.8 mmol) were added. The reaction was returned to heating and a solution of 4-iodo-2-(methylthio)pyrimidine (29.4 g, 117 mmol) in DMF (60 mL) was added via syringe pump over about 8 hours. The reaction was heated for about 15 hours after the addition ended then concentrated under reduced pressure. The resulting crude material was dissolved in DCM (600 mL) and washed with 1 N HCl (5×300 mL) followed by 0.5 N NaOH (300 mL) then filtered the resulting emulsion through Celite®. The layers of the filtrate were separated and the organic layer was washed with brine (2×300 mL), dried over MgSO₄, filtered, and concentrated under reduced pressure. The crude material was purified by silica gel chromatography (330 g column) using a gradient of DCM:ACN (1:0 for 4 min, ramped to 4:1 over 1 min, held for 35 min, ramped to 1:1 over 20 min and held for additional 5 min). The column fractions enriched in product were combined, concentrated under reduced pressure, triturated with IPA (75 mL), sonicated for 5 min then filtered, washing with additional IPA (10 mL) followed by petroleum ether (b.p. 30-60° C.), and dried in a vacuum oven at about 70° C. overnight to yield the title compound as a light brown solid (12.3 g, 51%): LC/MS (Table 1, Method b) Rₜ=2.25 min; MS m/z: 384.2 (M+H)⁺.

### Step D

### 2-(2,2-Difluorophenyl)-8-ethyl-3-(2-methanesulfonylpyrimidin-4-yl)-imidazo[1,2-α]pyrazine

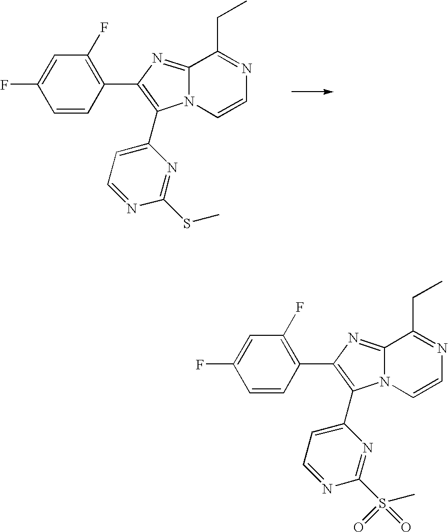

To a rapidly stirred solution of 2-(2,4-difluorophenyl)-8-ethyl-3-(2-methylsulfanylpyrimidin-4-yl)-imidazo[1,2-α]pyrazine (93% purity, 23.3 g, 56.5 mmol) in DCM (486 mL) and MeOH (486 mL) was added a solution of Oxone® (74.7 g, 122 mmol) in water (243 mL) at ambient temperature. After about 17 hours, the mixture was diluted with water (1.2 L) and extracted with DCM (3×300 mL). The combined organic layers were washed with brine (300 mL), dried over MgSO₄, filtered, and concentrated under reduced pressure. The crude material was purified by silica gel chromatography (330 g column) using a gradient of DCM:EtOAc (1:1 for 10 min, ramped to 0:1 over 30 min and held for additional 20 min). Fractions enriched in product were crystallized from hot ACN. The resulting solid was filtered, washing with additional ACN, and dried in a vacuum oven at about 70° C. overnight to give 9.85 g (42%) of the title compound. Additional product can be obtained from further purification of filtrate: LC/MS (Table 1, Method b) Rₜ=1.83 min; MS m/z: 416.1 (M+H)⁺.

### Step E

### 1-{4-[2-(2,4-Difluorophenyl)-8-ethylimidazo[1,2-α]pyrazin-3-yl]-pyrimidin-2-ylamino}-2-methylpropan-2-ol

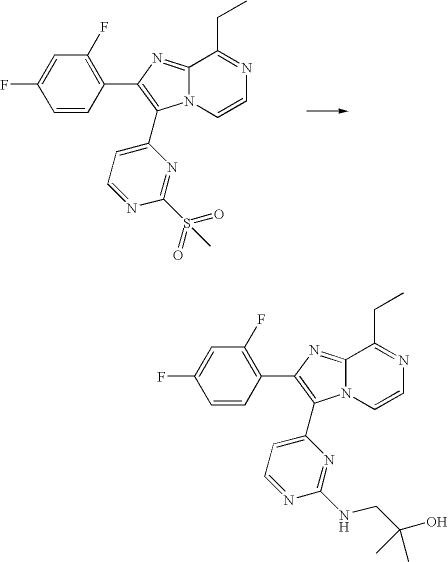

A mixture of 2-(2,4-difluorophenyl)-8-ethyl-3-(2-methanesulfonylpyrimidin-4-yl)-imidazo[1,2-a]pyrazine (2.53 g, 6.06 mmol) and 1-amino-2-methylpropan-2-ol (Tyger, 6.34 g, 71.1 mmol) in ACN (50 mL) was heated at about 85° C. overnight. The mixture was concentrated under reduced pressure and purified by silica gel chromatography using EtOAc as the eluent to give the title compound (1.97 g, 76%): LC/MS (Table 1, Method a) Rₜ=2.11 min; MS m/z: 425.2 (M+H)⁺, mp 164-165° C.

### Example #3

### 1-{4-[8-Cyclopropylmethyl-2-(2,4-difluorophenyl)-imidazo[1,2-α]pyrazin-3-yl]-pyrimidin-2-ylamino}-2-methylpropan-2-ol

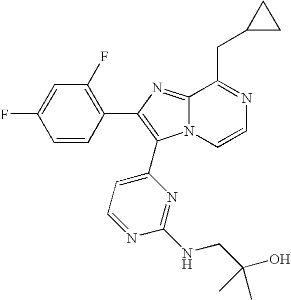

### Step A

### 8-Chloro-2-(2,4-difluorophenyl)-imidazo[1,2-α]pyrazine

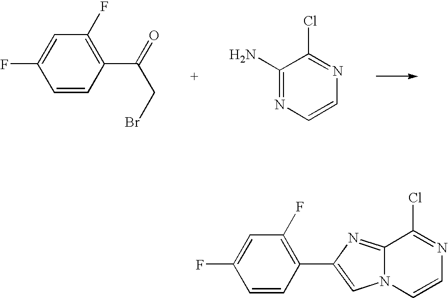

A mixture of 2-bromo-1-(2,4-difluorophenyl)ethanone (107.31 g, 442.89 mmol) and 3-chloropyrazin-2-amine (98.00 g, 756.5 mmol) in ACN (800 mL) was stirred at reflux for about 20 hours. The reaction mixture was cooled to about 25° C. before the resultant solid was collected. The filtrate solvent was removed in vacuo to yield a brown solid. This filtration was done to negate the bumping associated with the removal of the ACN. The combined solids were then suspended in water (750 mL) and basified, whilst stirring, with 2N NaOH (750 mL). After about 30 min, the product was partitioned between DCM (9×1000 mL) and filtered from the insoluble material to aid extraction process. The organic extracts were combined and stirred with 2.5N HCl (4×750 mL). The organic layer was finally washed with 2.0N NaOH (500 μL) and water (2×500 mL), dried over MgSO₄, and filtered through a Florisil® pad (3 inch diameter×3 inch depth) to remove origin material. The Florisil® pad was washed with repeated amounts of solvent until no product was detected by TLC. The organic solvent was removed in vacuo to yield a yellow solid. The solid was suspended in IPA (200 mL) at about 80° C. for about 15 min and then cooled to about 20° C. The solid was collected and washed with ice-cold IPA (2×40 mL), followed by petroleum ether [bp 30-60° C.] (3×80 mL) to remove impurities. The solid was dried in vacuo at about 70° C. overnight to yield the title compound as a yellow powdery solid (69.75 g, 57%): LC/MS (Table 1, Method a) Rₜ=2.70 min; MS m/z: 266.1 (M+H)⁺.

### Step B

### 8-Cyclopropylmethyl-2-(2,4-difluorophenyl)imidazo[1,2-a]pyrazine

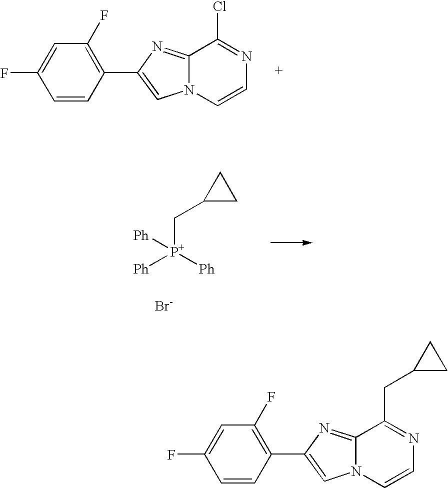

To a suspension of (cyclopropylmethyl)triphenylphosphonium bromide (Alfa Aesar, 165 g, 414 mmol) in anhydrous DME (800 mL) kept between about −30° C. and −40° C. was added a 2.5M solution of n-butyllithium in hexanes (166 mL, 414 mmol). After stirring between about −30° C. and −40° C. for about 1 h, 8-chloro-2-(2,4-difluorophenyl)imidazo[1,2-a]pyrazine (50.0 g, 188 mmol) was added rinsing in with DME (100 mL). The mixture was warmed to ambient temperature over about 1 hour, then heated at about 85° C. for about 2 hours, at which point Na₂CO₃ (21.94 g, 207 mmol) was added followed by the cautious addition of water (200 mL) and heating was continued for about 1.5 hours. The volatile solvents were removed under reduced pressure and to the resulting dark red reaction mixture was added EtOAc (750 mL) and H₂O (750 mL), stirred for about 10 min then the insoluble material was filtered and washed with EtOAc (3×100 mL). The aqueous phase separated and washed with EtOAc (3×150 mL). The combined organic extracts were washed with water (3×250 mL), dried over MgSO₄, filtered, and concentrated under reduced pressure to yield a dark red oil/gum. The residue was triturated with ether (4×250 mL), leaving the PPh₃O insoluble material, and the supernatant liquor was filtered through a Florisil® pad to also remove origin material. The solvent was removed under reduced pressure to yield a dark orange oil. The oil was triturated with heptane (3×200 mL) at about 60° C., allowed to cool to about 25-30° C., after which the supernatant liquid was decanted from the dark oil and further cooled to about 20° C. The resultant yellow solid was collected and washed with ice-cold petroleum ether [bp 30-60° C.] (2×15 mL) and dried to yield an initial batch of the title compound (17.3 g, 29%). The dark oil residue was dissolved in Et₂O (200 mL) and combined with the heptane filtrate. To this solution was added 5N HCl solution (400 mL). The suspension was stirred for about 1 hour and the HCl salt was filtered to yield a dark brown solid. This solid was stirred with ACN (2×60 mL), filtered, and dried to yield a yellow solid that was stirred with 1N NaOH (200 mL), water (200 mL) for about 10 min. The free base product was partitioned between EtOAc (4×150 mL) and the basic aqueous phase. The combined organic extracts were washed with water (3×250 mL), dried over MgSO₄, filtered, and concentrated under reduced pressure to yield additional title compound as a pale yellow powder (30.2 g, 56%): LC/MS (Table 1, Method b) Rₜ=2.26 min; MS m/z: 286.1 (M+H)⁺.

### Step C

### 8-(Cyclopropylmethyl)-2-(2,4-difluorophenyl)-3-(2-(methylthio)pyrimidin-4-yl)imidazo[1,2-a]pyrazine

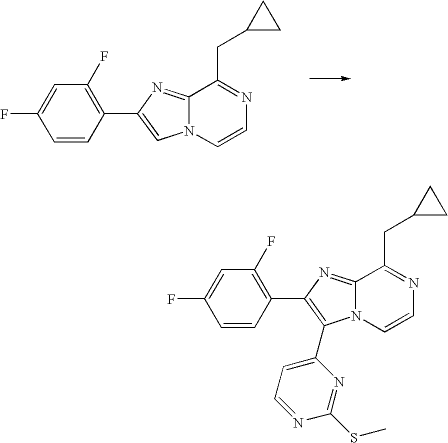

In a 500-mL round-bottomed flask equipped with rubber septum and nitrogen inlet needle was charged with PPh₃ (8.09 g, 30.8 mmol) and Pd(OAc)₂ (3.46 g, 15.4 mmol) in DMF (175 mL) to give a yellow suspension. The mixture was evacuated under vacuum and back-filled with nitrogen gas three times. The reaction mixture was heated at about 100° C. for about 10 min. CsOAc (29.6 g, 154 mmol) and 8-(cyclopropylmethyl)-2-(2,4-difluorophenyl)imidazo[1,2-a]pyrazine (22 g, 77 mmol) were each added sequentially in one portion to give a purple suspension. The mixture was evacuated under vacuum and back-filled with nitrogen gas three times then heated at about 100° C. for about 5 min. 4-iodo-2-(methylthio)pyrimidine (38.9 g, 154 mmol) in DMF (70 mL) was added dropwise via syringe pump over about 8 h to give a black suspension. The reaction mixture was heated at about 100° C. for about 20 hours. The reaction mixture was concentrated in vacuo, diluted with DCM, and washed with sequentially with H₂O, saturated aqueous NaCl, and H₂O (250 mL each). The organic layer was dried with MgSO₄, filtered through Celite®, and concentrated in vacuo. The crude material was purified by silica gel chromatography (330 g column) using a gradient of DCM:EtOAc (1:0 for 60 min, ramped to 1:1 over 60 min, held for 5 min). The product-containing column fractions were combined, concentrated under reduced pressure, and crystallized from hot EtOAc. The resulting needles were isolated by filtration, washing with petroleum ether (b.p. 30-60° C.), and dried in a vacuum oven at about 70° C. overnight to yield the title compound (15.2 g, 42%). Additional product can be obtained from further purification of filtrate: LC/MS (Table 1, Method b) Rₜ=2.4 min; MS m/z: 410.2 (M+H)⁺.

### Step D

### 8-(Cyclopropylmethyl)-2-(2,4-difluorophenyl)-3-(2-(methanesulfonyl)pyrimidin-4-yl)imidazo[1,2-a]pyrazine

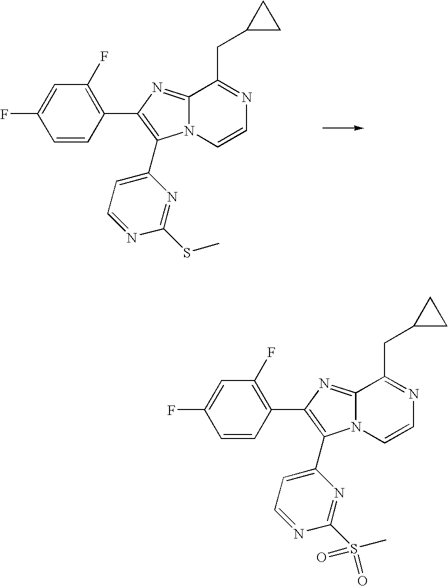

To a rapidly stirred solution of 8-(cyclopropylmethyl)-2-(2,4-difluorophenyl)-3-(2-(methylthio)pyrimidin-4-yl)imidazo[1,2-a]pyrazine (15.2 g, ˜87 wt %, 32.2 mmol) in DCM (200 mL) and MeOH (234 mL) was added a solution of Oxone® (39.6 g, 64.5 mmol) in water (123 mL) at ambient temperature. After about 16 hours, the mixture was diluted with water (150 mL) and DCM (200 mL). The layers were separated. The aqueous layer was extracted with DCM (3×100 mL). The combined organic layers were washed with brine (300 mL), dried over MgSO₄, filtered, and concentrated under reduced pressure. The crude material was purified by silica gel chromatography using a gradient of DCM:EtOAc (1:1 for 20 min, ramped to 0:1 over 20 min and held for additional 20 min). Fractions enriched in product were crystallized from hot ACN. The resulting solid was filtered, washing with ACN and petroleum ether (b.p. 30-60° C.), and dried in a vacuum oven at about 70° C. for about 5 hours to give 10.0 g (70%) of the title compound. Additional product can be obtained from further purification of filtrate: LC/MS (Table 1, Method b) Rₜ=2.0 min; MS m/z: 442.2 (M+H)⁺.

### Step E

### 1-{4-[8-Cyclopropylmethyl-2-(2,4-difluorophenyl)-imidazo[1,2-a]pyrazin-3-yl]-pyrimidin-2-ylamino}-2-methylpropan-2-ol

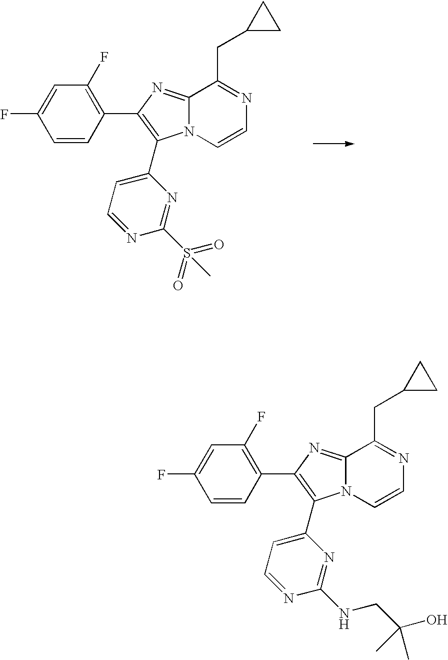

A mixture of 8-cyclopropylmethyl-2-(2,4-difluorophenyl)-3-(2-methanesulfonylpyrimidin-4-yl)imidazo[1,2-a]pyrazine (10.0 g, 22.6 mmol) and 1-amino-2-methylpropan-2-ol (Tyger, 6.06 g, 68.0 mmol) in ACN (162 mL) was heated to about 80° C. After about 16 hours, the mixture was concentrated under reduced pressure and purified by silica gel chromatography (120 g column) using a gradient of DCM/MeOH (1:0 for 5 min, ramped to 9:1 over 5 min, held for 20 min). The product-containing fractions were concentrated under reduced pressure to yield a white solid. Additionally, ˜3 g of a mixture of product and starting material was recovered and treated with 1-amino-2-methylpropan-2-ol (Tyger, 0.505 g, 5.66 mmol) in ACN (50 mL). The reaction mixture was heated at about 80° C. for about 5 hours then concentrated under reduced pressure to provide yellow solid. The yellow solid was purified by silica gel chromatography (120 g column) using a gradient of DCM/MeOH (1:0 for 10 min, ramped to 4:1 over 10 min, held for 10 min). The product-containing fractions were concentrated under reduced pressure then combined with the white solid from the first column by dissolving in MeOH. The solution was concentrated under reduced pressure until precipitate began to form. The suspension was sonicated until a uniform solid formed throughout then concentrated under reduced pressure and dried at about 100° C. under vacuum to yield the title compound as a white solid (9.5 g, 93%): LC/MS (Table 1, Method b) Rₜ=2.1 min; MS m/z: 451.2 (M+H)⁺, mp 174-176° C.

What is claimed is:

1. A compound of formula (1) 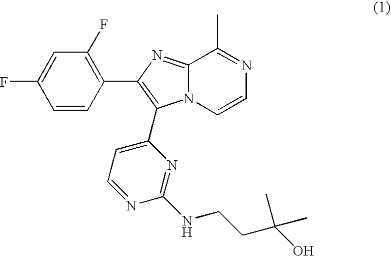
and pharmaceutically acceptable salts, prodrugs, and pharmaceutically active metabolites thereof.

2. A compound of formula (2) 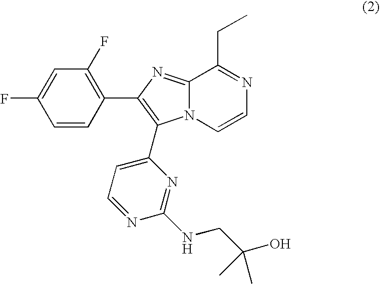
and pharmaceutically acceptable salts, prodrugs, and pharmaceutically active metabolites thereof.

3. A compound of formula (3) 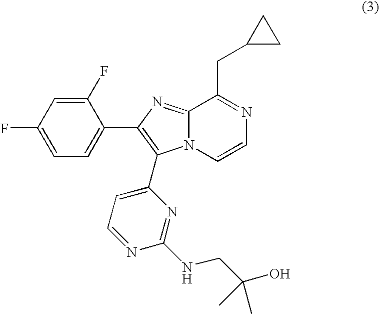
and pharmaceutically acceptable salts, prodrugs, and pharmaceutically active metabolites thereof.
